(19) United States
(12) Patent Application Publication
(10) Pub. No.: US 2008/0281428 A1
(43) Pub. Date: Nov. 13, 2008

(54) METHODS AND APPARATUSES FOR ATTACHING SOFT TISSUE TO ORTHOPAEDIC IMPLANTS

(75) Inventors: John E. Meyers, Columbia City, IN (US); Robert E. Montgomery, Mishawaka, IN (US); Dennis R. Aquilo, Youngsville, NC (US); Scott C. Lazar, Cromwell, IN (US); Douglas G. Branscome, Fort Wayne, IN (US); Natalia Fridshtand, Ringwood, NJ (US); Scott Cron, Wayne, NJ (US); Marvin Figueroa, Warsaw, IN (US); Clarence M. Panchison, Warsaw, IN (US); Kelly Timmons, Warsaw, IN (US); Doug Gabel, Pompton Plains, NJ (US); Douglas Wentz, Winona Lake, IN (US); Ron Donkers, Warsaw, IN (US); Greg Hippensteel, South Whitley, IN (US)

Correspondence Address: ZIMMER TECHNOLOGY - BAKER & DANIELS, 111 EAST WAYNE STREET, SUITE 800, FORT WAYNE, IN, 46802, US

(73) Assignee: ZIMMER, INC., Warsaw, IN (US)

(21) Appl. No.: 12/115,763

(22) Filed: May 6, 2008

Related U.S. Application Data
(60) Provisional application No. 60/916,414, filed on May 7, 2007.

(51) Int. Cl. A61F 2/38

(52) U.S. Cl. 623/20.35; 623/20.32

(57) ABSTRACT

Methods and apparatuses for attaching soft tissue and/or bone to orthopaedic implants. In one exemplary embodiment, the methods and apparatuses are used to attach soft tissue and/or bone to a proximal tibial implant. In another exemplary embodiment, the methods and apparatuses are used to attach soft tissue and/or bone to a proximal femoral implant.

### CROSS-REFERENCE TO RELATED APPLICATIONS

This application claims the benefit under Title 35 U.S.C. §119(e) of U.S. Provisional Patent Application Ser. No. 60/916,414, entitled METHODS AND APPARATUSES FOR ATTACHING SOFT TISSUE TO ORTHOPAEDIC IMPLANTS, filed on May 7, 2007, the entire disclosure of which is expressly incorporated by reference herein.

### BACKGROUND

1. Field of the Disclosure

The present disclosure relates to methods and apparatuses for attaching soft tissue to orthopaedic implants. More particularly, the present disclosure relates to methods and apparatuses for attaching soft tissue to a proximal tibial implant and a proximal femoral implant.

2. Description of the Related Art

Orthopaedic implants are commonly used to replace at least a portion of a patient's joint in order to restore the use of the joint, or to increase the use of the joint, following deterioration due to aging or illness, injury due to trauma, or disease.

### SUMMARY

The present disclosure provides methods and apparatuses for attaching soft tissue and/or bone to orthopaedic implants. In one exemplary embodiment, the methods and apparatuses are used to attach soft tissue and/or bone to a proximal tibial implant. In another exemplary embodiment, the methods and apparatuses are used to attach soft tissue and/or bone to a proximal femoral implant.

### BRIEF DESCRIPTION OF THE DRAWINGS

The above-mentioned and other features of the disclosure, and the manner of attaining them, will become more apparent and will be better understood by reference to the following description of embodiments of the disclosure taken in conjunction with the accompanying drawings, wherein:

FIG. 1 is a perspective view of a proximal tibial implant according to an exemplary embodiment of the present disclosure;

FIG. 2 is another perspective view of the proximal tibial implant of FIG. 1;

FIG. 3 is a side view of the proximal tibial implant of FIG. 1;

FIG. 4 is an anterior view of the proximal tibial implant of FIG. 1;

FIG. 5 is another side view of the proximal tibial implant of FIG. 1;

FIG. 6 is an anterior view of the proximal tibial implant of FIG. 1, further illustrating a rotating fixation structure;

FIG. 7 is a cross-sectional view of an exemplary embodiment of the rotating fixation structure of FIG. 6;

FIG. 8 is a cross-sectional view of another exemplary embodiment of the rotating fixation structure of FIG. 6;

FIG. 9 is an anterior view of a proximal tibial implant according to another exemplary embodiment of the present disclosure;

FIG. 10 is a partially exploded perspective view of a proximal tibial implant according to yet another exemplary embodiment of the present disclosure;

FIG. 11 is a perspective view of a proximal tibial implant according to an exemplary embodiment of the present disclosure;

FIG. 12 is a cross-sectional view of a portion of the proximal tibial implant of FIG. 11, taken along line 12-12 of FIG. 11;

FIG. 13 is a perspective view of a proximal femoral implant according to an exemplary embodiment of the present disclosure;

FIG. 14 is another perspective view of the proximal femoral implant of FIG. 13;

FIG. 15 is an anterior/posterior view of the proximal femoral implant of FIG. 13;

FIG. 16 is a medial view of the proximal femoral implant of FIG. 13;

FIG. 17 is an inferior view of the proximal femoral implant of FIG. 13;

FIGS. 18-21 are various views of another embodiment proximal tibial implant according to the present disclosure; and

FIG. 22 is a view of another embodiment proximal femoral implant according to the present disclosure.

Corresponding reference characters indicate corresponding parts throughout the several views. The exemplifications set out herein illustrate embodiments of the disclosure and such exemplifications are not to be construed as limiting the scope of the invention in any manner.

### DETAILED DESCRIPTION

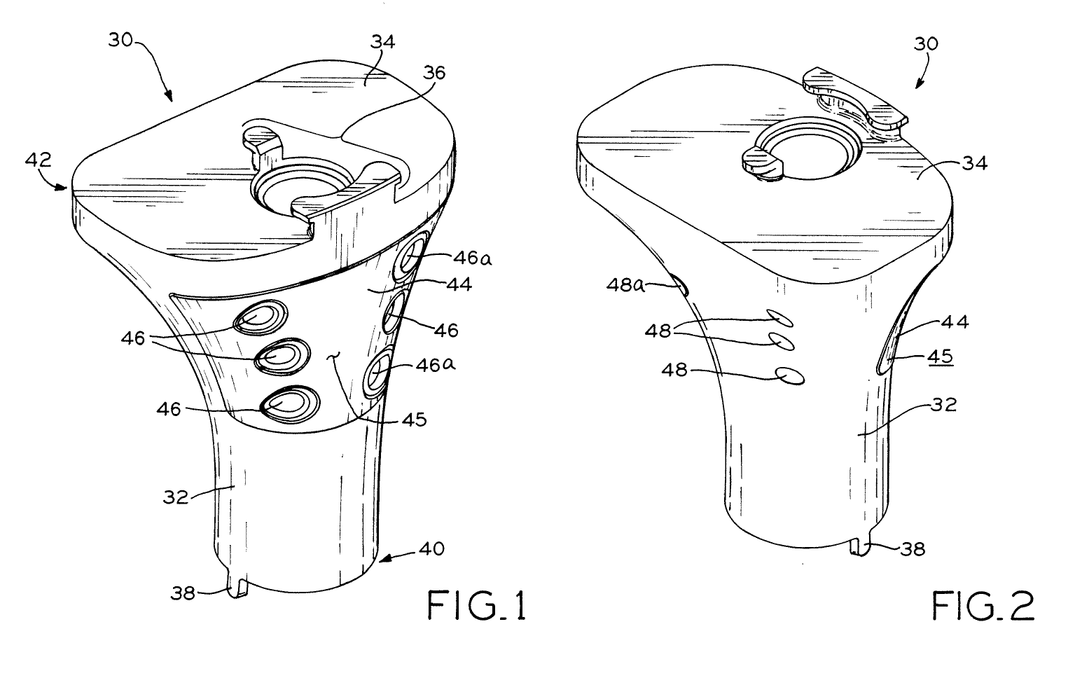

Referring now to FIGS. 1 and 2, proximal tibial implant 30 is shown and may be used to restore mechanical and biological fixation of soft tissue structures, such as muscle, ligaments, and/or tendons, for example, associated with a knee joint of a patient to enhance the stability of the knee joint and to restore knee joint kinematics. Proximal tibial implant 30 may typically be used in a patient requiring complete metaphyseal removal of the proximal tibia. Proximal tibial implant 30 may include body 32 having proximal end 42 and distal end 40, tibial plate 34, and mating structure 36.

Body 32 may be formed from relatively light-weight material, such as titanium, a cobalt chromium alloy, or other suitable biocompatible material, for example, thereby making it easier for the patient to lift and extend the knee joint, particularly in procedures which require extensive removal of muscle proximate the knee joint. In one embodiment, tibial plate 34 may be formed as a modular component of proximal tibial implant 30 to provide more interoperative options. In an exemplary embodiment, tibial plate 34 is formed as a wear-resistant tibial plate to minimize debris from articulation with another component (not shown) of the knee joint. Mating structure 36 may be formed complementary to a meniscal component (not shown) of the knee joint to provide either a mobile or a non-mobile bearing connection between proximal tibial implant 30 and the meniscal component. Proximal tibial implant 30 may also include rotational adjustment tabs 38 at distal end 40 of proximal tibial implant 30 to allow for in vivo rotational adjustment of proximal tibial implant 30 relative to another implant or to the remaining structure of the tibia. Rotational adjustment tabs 38 may generally extend distally from body 32 along a lateral and/or a medial side of implant 30. A plurality of tabs 38 may be utilized or a single tab 38 may be utilized.

In an exemplary embodiment, proximal tibial implant 30 also includes soft tissue attachment plate 44 including a plurality of fastener or suture apertures 46. Attachment plate 44 may be positioned on an anterior surface of proximal tibial implant 30 and may be integrally formed with proximal tibial implant 30. In one embodiment, attachment plate 44 is formed as a modular component of proximal tibial implant 30. In another embodiment, attachment plate 44 is positioned in a recess (not shown) provided on the anterior surface of proximal tibial implant 30. Attachment plate 44 may generally have a relatively thin anterior to posterior thickness, e.g., as low as approximately 1 mm to as high as approximately 5 mm, such as to define a relatively slim profile such that attachment plate 44 does not protrude from proximal tibial implant 30 and consequently potentially interfere with other anatomical structures. For example, in an exemplary embodiment, the anterior surface of attachment plate 44 is substantially flush with the remainder of the anterior surface of proximal tibial implant 30. Apertures 46 may be generally aligned with respective throughbores 48 (FIG. 2) which extend through proximal tibial implant 30 from the anterior surface to a posterior surface. Attachment plate 44 provides a direct connection between a soft tissue structure and proximal tibial implant 30. For example, a patella tendon, which joins a lower edge of a patella (not shown) of the knee joint with a tibial tubercle of a tibia, may be directly attached via mechanical and/or biological fixation to proximal tibial implant 30 after implantation of implant 30 via attachment plate 44. Such fixation of the patella tendon to proximal tibial implant 30 enhances usability of the knee joint. For example, when a patient jumps into the air or allows the tibia to hang without any support, the fixation of the patella tendon to proximal tibial implant 30 prevents dislocation of the components of the prosthetic knee joint and facilitates normal functioning of the prosthetic knee joint after the jump or once the tibia is again supported. In one example, a rotating hinged knee includes a femoral component with a post extending through a meniscal component and into a tibial component. During a jump or when the tibia is unsupported, the patella tendon effectively prevents the post from extending too far from the tibial component and ensures that the post returns to proper engagement with the tibial component once normal functioning is resumed.

As described further below, attachment plate 44 may include at least one porous surface 45, such as a surface to facilitate ingrowth of soft tissues. In one embodiment, porous surface 45 may be formed of a material having a cellular structure which resembles bone and approximates the physical and mechanical properties of bone, thereby enabling rapid and extensive soft tissue infiltration and strong attachment of soft tissue structures thereto. For example, the material may be a highly porous biomaterial having a porosity as low as 55, 65, or 75 percent and as high as 80, 85, or 90 percent. An example of such a material is produced using Trabecular Metal™ technology generally available from Zimmer, Inc., of Warsaw, Ind. Trabecular Metal™ is a trademark of Zimmer Technology, Inc. Such a material may be formed from a reticulated vitreous carbon foam substrate which is infiltrated and coated with a biocompatible metal, such as tantalum, etc., by a chemical vapor deposition (“CVD”) process in the manner disclosed in detail in U.S. Pat. No. 5,282,861, the disclosure of which is expressly incorporated herein by reference. In addition to tantalum, other metals such as niobium, or alloys of tantalum and niobium with one another or with other metals may also be used.

Generally, the porous tantalum structure includes a large plurality of ligaments defining open spaces therebetween, with each ligament generally including a carbon core covered by a thin film of metal such as tantalum, for example. The open spaces between the ligaments form a matrix of continuous channels having no dead ends, such that growth of cancellous bone through the porous tantalum structure is uninhibited. The porous tantalum may include up to 75%-85% or more void space therein. Thus, porous tantalum is a lightweight, strong porous structure which is substantially uniform and consistent in composition, and closely resembles the structure of natural cancellous bone. The porous tantalum structure may be made in a variety of densities in order to selectively tailor the structure for particular applications. In particular, as discussed in the above-incorporated U.S. Pat. No. 5,282,861, the porous tantalum may be fabricated to virtually any desired porosity and pore size, and can thus be matched with the surrounding natural bone in order to provide an improved matrix for bone ingrowth and mineralization. Such porous material facilitates ingrowth of soft tissue for enhanced attachment of soft tissue structures to proximal tibial implant 30. For example, struts which extend from porous surface 45 are generally rough which facilitates holding a soft tissue structure in such a manner that damage and disengagement of the soft tissue structure is discouraged. The porous material may have a generally corrugated surface to further facilitate biological fixation of soft tissue structures thereto.

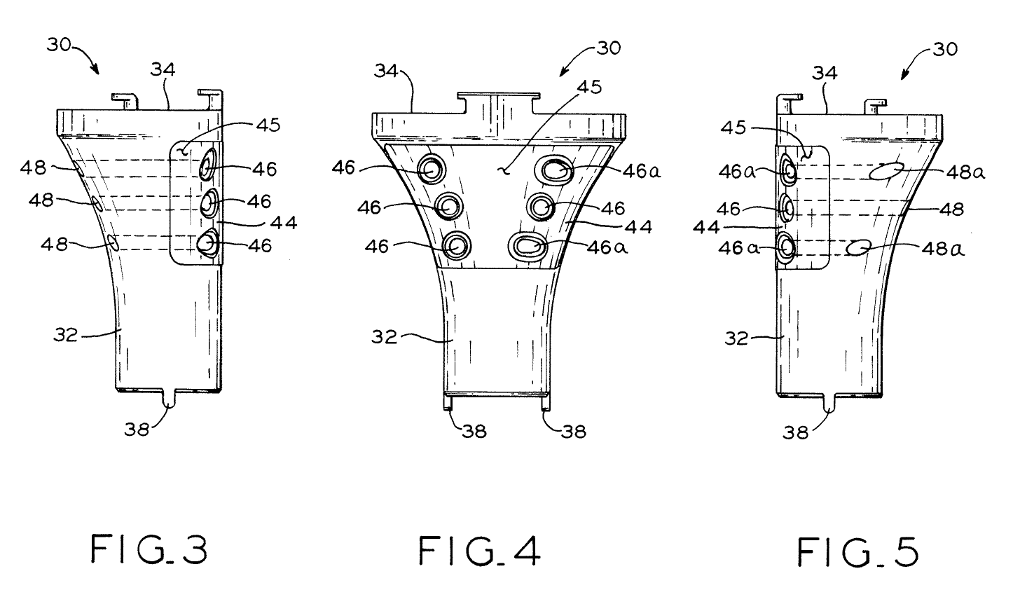

As shown in FIGS. 2-5, throughbores 48 generally extend through proximal tibial implant 30 from the anterior surface to the posterior surface. Apertures 46 may include apertures 46a which are generally skewed such that throughbores 48a corresponding to apertures 46a formed a curved path through body 32, as opposed to a straight path through body 32 formed by throughbores 48. Apertures 46, 46a may have a generally rounded shape to facilitate prevention of suture or fastener breakage during an operation.

In operation, a surgeon may attach a soft tissue structure, such as muscle, a ligament, a capsule, and/or a tendon, for example, and/or bone to proximal tibial implant 30. The surgeon positions the soft tissue structure in contact with the anterior surface of attachment plate 44. At least one suture is then threaded through a plurality of apertures 46, 46a and throughbores 48, 48a to maintain contact between the soft tissue structure and attachment plate 44. In contrast to sutures, a surgeon may also use surgical tape or surgical cables, for example. The surgeon may select any or all apertures 46, 46a and throughbores 48, 48a to at least temporarily secure the soft tissue structure to proximal tibial implant 30. In one embodiment, the sutures may be biodegradable after a period of time in which the soft tissue structure is permanently attached to proximal tibial implant 30 via ingrowth of the soft tissue structure into porous surface 45 of attachment plate 44. The close proximity of apertures 46, 46a in relation to contact between the soft tissue structure and attachment plate 44 further facilitates more direct contact between the soft tissue structure and attachment plate 44. Alternatively, the soft tissue structure may be first attached to attachment plate 44 and then attachment plate 44 is attached to body 32, thereby providing potential for tensioning the soft tissue structure prior to securement of attachment plate 44 to body 32.

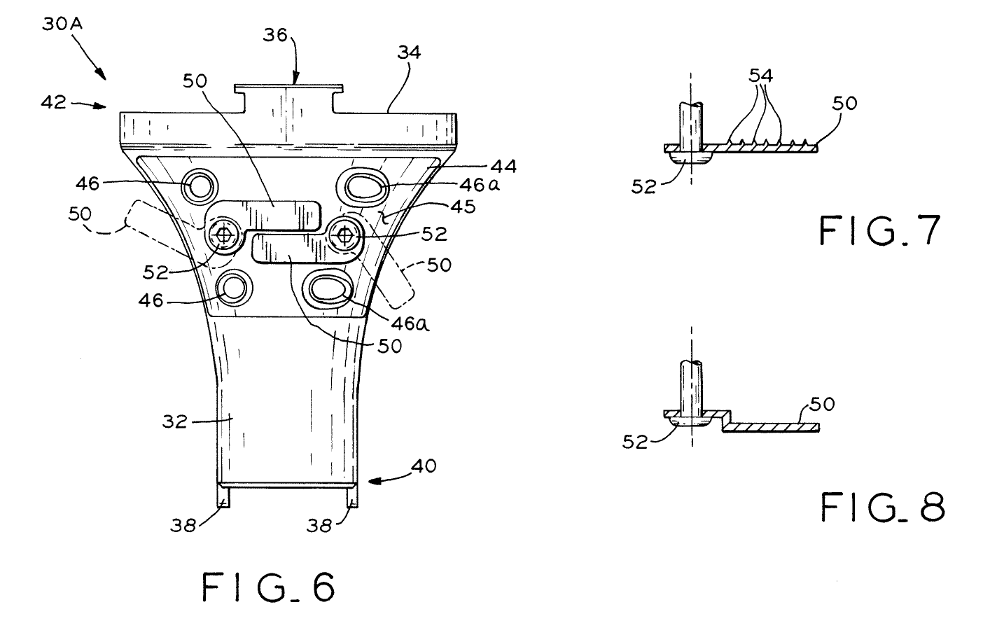

Referring now to FIGS. 6-8, an alternative embodiment proximal tibial implant 30A is shown and is substantially identical to proximal tibial implant 30, described above with reference to FIGS. 1-5, except as described below. Proximal tibial implant 30A includes body 32, tibial plate 34, mating structure 36, rotational adjustment tabs 38, distal end 40, proximal end 42, and attachment plate 44. Proximal tibial implant 30A may include surgical fasteners such as washers 50 which are attached to attachment plate 44 via fasteners 52 inserted through any apertures 46 provided in attachment plate 44. Washers 50 may be rotatably attached to attachment plate 44 such that washers 50 are rotatable from a first position, shown in dashed lines in FIG. 6, in which a soft tissue structure is not engaged with washers 50, to a second position, shown in solid lines in FIG. 6, in which a soft tissue structure may be securely attached to attachment plate 44 with washers 50 on an anterior surface of proximal tibial implant 30A. In one embodiment, as shown in FIG. 7, washer 50 may include a plurality of teeth 54 to further facilitate holding engagement of the soft tissue structure with washer 50. In another embodiment, as shown in FIG. 8, washer 50 may include a porous material similar to porous surface 45 on attachment plate 44, as described above, to facilitate ingrowth of the soft tissue structure into washer 50, thereby facilitating secure holding engagement between washer 50 and the soft tissue structure.

In operation, a surgeon may attach a soft tissue structure, such as a muscle, a ligament, a capsule, and/or a tendon, for example, and/or bone to proximal tibial implant 30A. The surgeon positions the soft tissue structure in contact with the anterior surface of attachment plate 44. At least one optional washer 50 may then be moved from the first position into the second position to mechanically fixate the soft tissue structure with attachment plate 44. Each washer 50 may include a locking mechanism to lock washer 50 in the second position. In an exemplary procedure, a surgeon may pull or otherwise manipulate a soft tissue structure in close proximity to attachment plate 44, such as a portion of a calf muscle, for example, and use a washer 50 to pull and engage such soft tissue structure into contact with porous surface 45 of attachment plate 44 such that the soft tissue structure is fixated to proximal tibial implant 30A via washer 50. Washers 50 may be positioned to either pull such a soft tissue structure from either a medial or a lateral side of proximal tibial implant 30A. Alternatively, instead of washers 50, a surgeon may suture tissue to tissue to create a “sling” around implant 30A.

Also, at least one suture may be threaded through a plurality of apertures 46, 46a and throughbores 48, 48a to further maintain contact between the soft tissue structure and attachment plate 44. The surgeon may select any or all apertures 46, 46a and throughbores 48, 48a to secure the soft tissue structure to proximal tibial implant 30A. In one embodiment, the sutures may be biodegradable after a period of time in which the soft tissue structure is permanently attached to proximal tibial implant 30A via ingrowth of the soft tissue structure into porous surface 45 of attachment plate 44. The close proximity of apertures 46, 46a in relation to contact between the soft tissue structure and attachment plate 44 further facilitates more direct contact between the soft tissue structure and attachment plate 44 and enhances the ingrowth of the soft tissue structure into porous surface 45. Moreover, washers 50 provide a mechanical fixation between the soft tissue structure and proximal tibial implant 30A. Thus, the soft tissue structure may be both biologically and mechanically affixed to proximal tibial implant 30A. Alternatively, the soft tissue structure may be first attached to attachment plate 44 and then attachment plate 44 is attached to body 32.

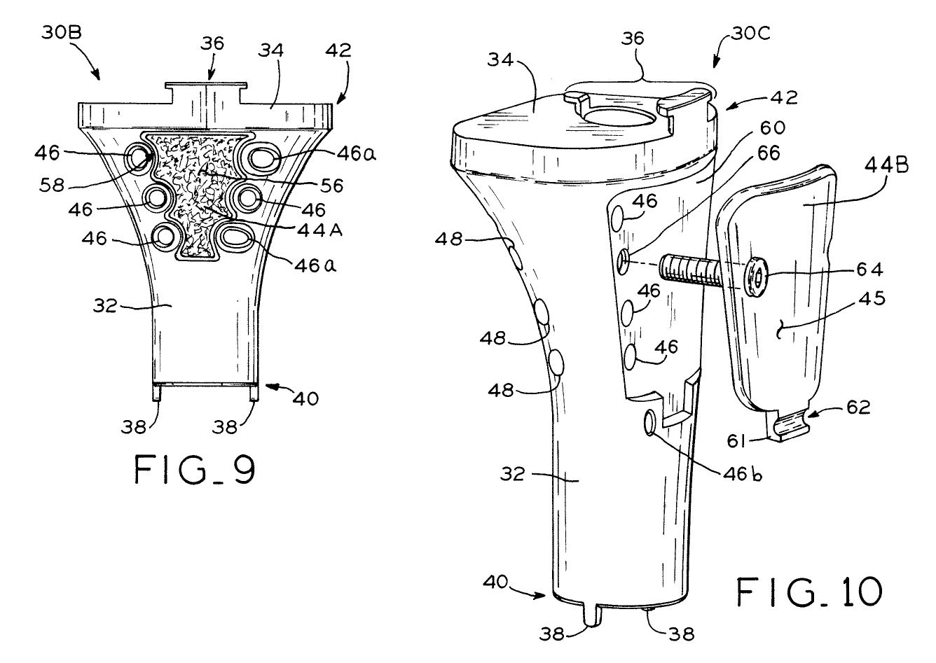

Referring now to FIG. 9, another embodiment proximal tibial implant 30B is shown and may be substantially identical to proximal tibial implant 30, described above with reference to FIGS. 1-5, or proximal tibial implant 30A, described above with reference to FIGS. 6-8, except as described below. Proximal tibial implant 30B includes body 32, tibial plate 34, mating structure 36, rotational adjustment tabs 38, distal end 40, and proximal end 42. Proximal tibial implant 30B may include attachment plate 44A which is substantially identical to attachment plate 44, described above with reference to FIGS. 1-5, except as described below. Attachment plate 44A includes porous surface 56 which is substantially identical to porous surface 45 on attachment plate 44. Porous surface 56 provides a porous surface on the anterior surface of proximal tibial implant 30B to facilitate ingrowth of a soft tissue structure. Apertures 46, 46a are provided in body 32, as opposed to being provided in attachment plate 44A, thereby eliminating any potential contact of the porous material on porous surface 56 with apertures 46, 46a. Attachment plate 44A includes contours 58 which substantially mimic the shape of apertures 46, 46a and extend attachment plate 44A adjacent to and in close proximity to each aperture 46, 46a.

In operation, a surgeon may attach a soft tissue structure, such as a muscle, a ligament, a capsule, and/or a tendon, for example, and/or bone to proximal tibial implant 30B. The surgeon positions the soft tissue structure in contact with the anterior surface of attachment plate 44A. At least one suture is then threaded through a plurality of apertures 46, 46a and throughbores 48, 48a to maintain contact between the soft tissue structure and attachment plate 44A. The surgeon may select any or all apertures 46, 46a and throughbores 48, 48a to secure the soft tissue structure to proximal tibial implant 30B. In one embodiment, the sutures may be biodegradable after a period of time in which the soft tissue structure is permanently attached to proximal tibial implant 30B via ingrowth of the soft tissue structure into porous surface 56 of attachment plate 44A. The close proximity of apertures 46, 46a in relation to attachment plate 44A further facilitates more direct contact between the soft tissue structure and attachment plate 44A. Alternatively, the soft tissue structure may be first attached to attachment plate 44A and then attachment plate 44A is attached to body 32.

Referring now to FIG. 10, yet another embodiment proximal tibial implant 30C is shown and may be substantially identical to proximal tibial implant 30, described above with reference to FIGS. 1-5, proximal tibial implant 30A, described above with reference to FIGS. 6-8, and proximal tibial implant 30B, described above with reference to FIG. 9, except as described below. Proximal tibial implant 30C includes body 32, tibial plate 34, mating structure 36, rotational adjustment tabs 38, distal end 40, and proximal end 42. Proximal tibial implant 30C may include attachment plate 44B which is substantially identical to attachment plate 44, described above with reference to FIGS. 1-6, and attachment plate 44A, described above with reference to FIG. 9, except as described below. Attachment plate 44B may be a modular component and attached to body 32 via fastener 64 inserted through attachment plate 44B and into a corresponding aperture 66 in body 32. Fastener 64 may be a screw, bolt, anchor mechanism, a mechanism which expands once inserted into body 32, or any other suitable fastening mechanism. Body 32 may include throughbores 48, apertures 46, and recess 60 formed in an anterior surface of body 32. Recess 60 is sized and configured to accept attachment plate 44B in a close-fitting engagement and to provide clearance for extension 61 of attachment plate 44B which includes groove 62. In one embodiment, attachment plate 44B may include a smooth posterior surface to facilitate the close-fitting engagement with recess 60. In another embodiment, the posterior surface of attachment plate 44B and/or the surface of recess 60 may include a porous surface substantially identical to porous surface 45 on the anterior surface of attachment plate 44B.

In operation, a surgeon may attach a soft tissue structure, such as a muscle, a ligament, a capsule, and/or a tendon, for example, and/or bone to proximal tibial implant 30C. The surgeon positions the soft tissue structure in contact with the anterior surface of attachment plate 44B. At least one suture is then threaded through a plurality of apertures 46, 46a and throughbores 48, 48a to maintain contact between the soft tissue structure and attachment plate 44B. The surgeon may select any or all apertures 46, 46a and throughbores 48, 48a to secure the soft tissue structure to proximal tibial implant 30C. Furthermore, the surgeon may thread at least one suture through groove 62 and around extension 61 of attachment plate 44B and through distal aperture 46b in body 32 to further facilitate mechanical fixation and stabilization of either or both of attachment plate 44B to body 32 and/or the soft tissue structure to proximal tibial implant 30C. In one embodiment, the sutures may be biodegradable after a period of time in which the soft tissue structure is permanently attached to proximal tibial implant 30C via ingrowth of the soft tissue structure into porous surface 45 of attachment plate 44B. The close proximity of apertures 46, 46a, 46b in relation to contact between the soft tissue structure and attachment plate 44B further facilitates more direct contact between the soft tissue structure and attachment plate 44B, thereby enhancing the ingrowth of the soft tissue structure into porous surface 45. Alternatively, the soft tissue structure may be first attached to attachment plate 44B and then attachment plate 44B is attached to body 32. In another embodiment, the soft tissue structure is positioned between attachment plate 44B and body 32 and then attachment plate 44B is attached to body 32 via fastener 64. A porous surface on the posterior surface of attachment plate 44B and on the surface of recess 60 facilitates ingrowth of the soft tissue structure into proximal tibial implant 30C. In one embodiment, attachment plate 44B is formed of a material which allows soft tissue and blood vessels to grow through the plate. In this manner, attachment plate 44B may secure a soft tissue structure, such as the patellar tendon, to implant 30C and then a muscle, such as a calf muscle, may be wrapped over the anterior surface of attachment plate 44B to facilitate blood flow through attachment plate 44B, thereby reducing the potential of subcutaneous irritation and necrosis of the soft tissue structure.

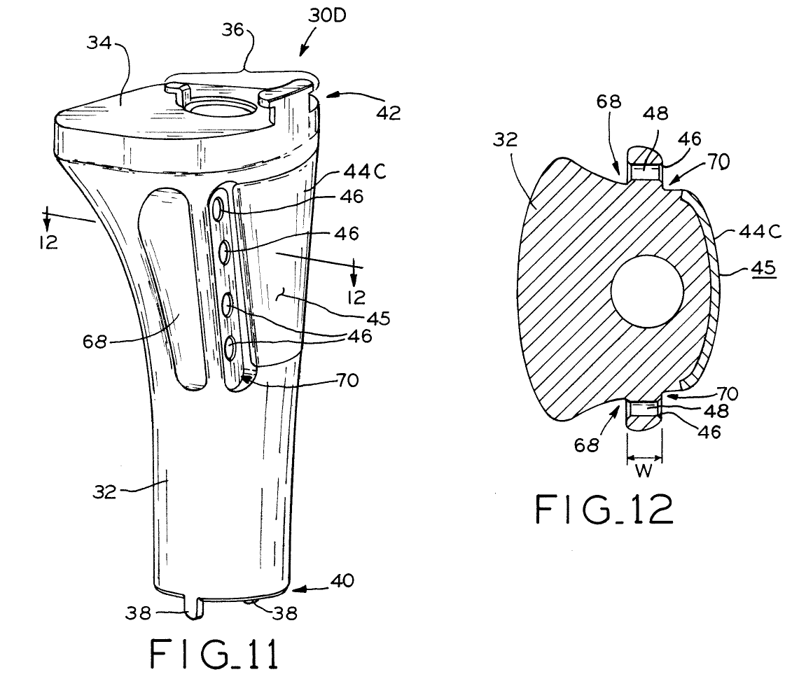

Referring now to FIGS. 11 and 12, an exemplary embodiment proximal tibial implant 30D is shown and may be substantially identical to proximal tibial implant 30, described above with reference to FIGS. 1-5, proximal tibial implant 30A, described above with reference to FIGS. 6-8, proximal tibial implant 30B, described above with reference to FIG. 9, and proximal tibial implant 30C, described above with reference to FIG. 10, except as described below. Proximal tibial implant 30D includes body 32, tibial plate 34, mating structure 36, rotational adjustment tabs 38, distal end 40, and proximal end 42. Proximal tibial implant 30D may include attachment plate 44C which may be substantially identical to attachment plate 44, described above with reference to FIGS. 1-6, attachment plate 44A, described above with reference to FIG. 9, and attachment plate 44B, described above with reference to FIG. 10, except as described below. Attachment plate 44C may be a modular component and attached to body 32 via suitable fasteners (not shown), an adhesive material, an interference fit, or, alternatively, attachment plate 44C may be integrally formed with body 32. Body 32 may include apertures 46, throughbores 48, recess 68, and recess 70. Recesses 68, 70 may be formed on either or both medial or lateral side of body 32. Recesses 68 may be formed toward the posterior side of body 32 but maintain proximity to the anterior side of body 32. Recesses 70 may be formed on the anterior side of body 32 adjacent to attachment plate 44C. Apertures 46 may be positioned in recesses 70 and throughbores 48 may extend from each recess 70 to a corresponding recess 68, such that throughbore 48 defines width W.

In operation, a surgeon may attach a soft tissue structure, such as a muscle, a ligament, a capsule, and/or a tendon, for example, and/or bone to proximal tibial implant 30D. The surgeon positions the soft tissue structure in contact with the anterior surface of attachment plate 44C. At least one suture is then threaded through a plurality of apertures 46 and throughbores 48 to maintain contact between the soft tissue structure and attachment plate 44C. The surgeon may select any or all apertures 46 and throughbores 48 to secure the soft tissue structure to proximal tibial implant 30D. In one embodiment, the sutures may be biodegradable after a period of time in which the soft tissue structure is permanently attached to proximal tibial implant 30D via ingrowth of the soft tissue structure into porous surface 45 of attachment plate 44C. The close proximity of apertures 46 in relation to attachment plate 44C further facilitates more direct contact between the soft tissue structure and attachment plate 44C. Moreover, recesses 68, 70 facilitate easier suture passage through apertures 46 and throughbores 48 and the thinness of width W of throughbore 48 enhances the ability to pass a curved suture needle therethrough. Alternatively, the soft tissue structure may be first attached to attachment plate 44C and then attachment plate 44C is attached to body 32.

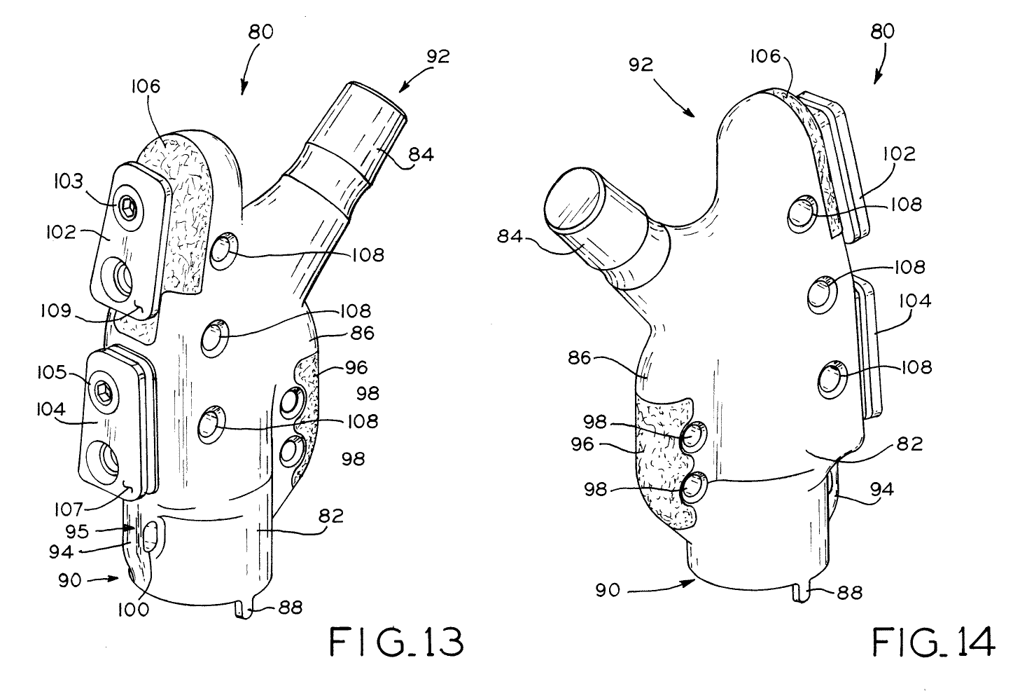

Referring now to FIGS. 13-17, proximal femoral implant 80 is shown and may be used to restore mechanical and biological fixation of soft tissue associated with a hip joint of a patient to enhance the stability of the hip joint, to restore hip joint function, and to enhance hip joint kinematics. Proximal femoral implant 80 may typically be used in a patient requiring complete metaphyseal removal of the proximal femur. Proximal femoral implant 80 may include body 82 including proximal end 92 and distal end 90. Proximal femoral implant 80 may also include neck 84 configured to mate with a corresponding femoral head component (not shown) of a prosthetic hip joint. The femoral head component may articulate against a natural or a prosthetic acetabulum. Proximal femoral implant 80 may also include anteversion adjustment tabs 88 at distal end 90 to allow for in vivo rotational adjustment of proximal femoral implant 80 relative to a prosthetic femoral stem component or the natural femur. Anteversion adjustment tabs 88 may generally extend distally from body 82 along a lateral and/or a medial side of implant 80. A plurality of tabs 88 may be utilized or a single tab 88 may be utilized.

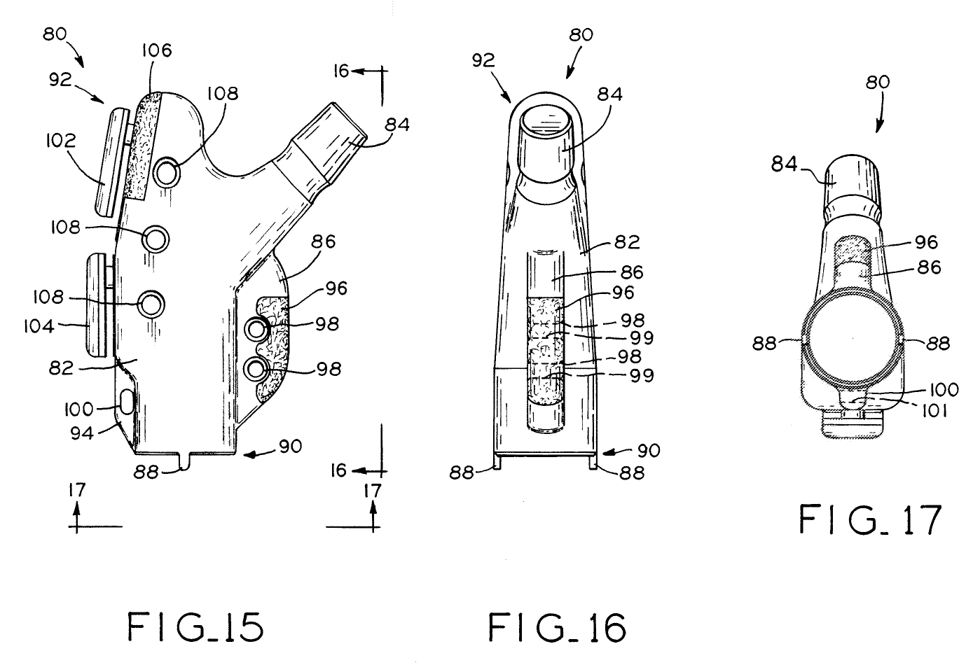

Proximal femoral implant 80 may include medial protrusion 86 and lateral protrusion 94. Medial protrusion 86 may include porous surface 96 and a plurality of apertures 98 defining throughbores 99 (FIG. 16). Porous surface 96 may be an inlay of porous material substantially identical to porous surface 45, described above. Porous surface 96 may be formed integrally with medial protrusion 86 or, alternatively, porous surface 96 may be formed on a plate which is attached to medial protrusion 86. Medial protrusion 86 may also buttress neck 84 and provide additional support to proximal femoral implant 80 when implanted into a hip joint. Lateral protrusion 94 may include aperture 100 defining throughbore 101 (FIG. 17).

Proximal femoral implant 80 may include lateral ingrowth pads 102, 104 each including porous surface 109, 107, respectively, and attached to body 82 via fasteners 103, 105, respectively. Body 82 may also include lateral proximal porous surface 106 and a plurality of lateral apertures 108 which may define throughbores which extend through body 82 from an anterior side to a posterior side. Body 82 may also include region 95 of porous material which is bonded to body 82. Porous surfaces 106, 109, 107, 95 may be substantially similar to porous surface 45, described above with respect to FIGS. 1-5. Porous surface 106 may substantially wrap around the proximal lateral end of body 82 from an anterior to a posterior side of body 82 to provide optimal attachment surfaces for soft tissue and bone structures, as described below.

As shown in FIG. 15, lateral ingrowth pad 102 may be slightly angled, i.e., the proximal end of lateral ingrowth pad 102 is more medially positioned than the distal end of lateral ingrowth pad 102. The angle of lateral ingrowth pad 102 facilitates prevention of potential irritation of subcutaneous tissue surrounding the proximal lateral portion of proximal femoral implant 80 after implantation. Furthermore, as shown in FIG. 16, proximal femoral implant 80 defines a generally slender profile with no protruding edges or structures in the anterior or posterior directions. The slender profile also tapers proximally toward proximal end 92 from distal end 90 which facilitates soft tissue closure after a procedure is completed and prevention of potential damage to subcutaneous tissue.

In operation, a surgeon may attach a plurality of soft tissue structures, such as a muscle, a ligament, a capsule, and/or a tendon, for example, and/or bone to proximal femoral implant 80.

To attach a soft tissue structure to the medial side of proximal femoral implant 80, a surgeon positions the soft tissue structure in contact with porous surface 96 on medial protrusion 86. At least one suture or other surgical fastener, such as surgical tape or surgical cable, is then threaded through one or both apertures 98 and through throughbores 99 to secure the soft tissue structure to implant 80 and to maintain contact between the soft tissue structure and porous surface 96. In one embodiment, the sutures may be biodegradable after a period of time in which the soft tissue structure is permanently attached to proximal femoral implant 80 via ingrowth of the soft tissue structure into porous surface 96. The close proximity of apertures 98 in relation to porous surface 96 further facilitates more direct contact between the soft tissue structure and porous surface 96, thereby enhancing ingrowth of the soft tissue structure into porous surface 96. In an exemplary embodiment, the soft tissue structure is an iliopsoas muscle which, when attached to proximal femoral implant 80, enhances stability of the hip joint.

To attach a soft tissue structure to the lateral side of implant 80, the surgeon positions the soft tissue structure in contact with one or more of porous surfaces 106, 109, and/or 107. At least one suture or other surgical fastener is then threaded through at least one aperture 108 and the associated throughbore proximate to the soft tissue structure attachment to secure the soft tissue structure to proximal femoral implant 80 and to maintain contact between the soft tissue structure and porous surface 106, 109, and/or 107. In one embodiment, the sutures may be biodegradable after a period of time in which the soft tissue structure is permanently attached to proximal femoral implant 80 via ingrowth of the soft tissue structure into either or all of porous surfaces 106, 109, and/or 107. The close proximity of apertures 108 in relation to porous surfaces 106, 107, 109 further facilitates more direct contact between the soft tissue structure and porous surfaces 106, 107, 109. In an exemplary embodiment, the soft tissue structure is an abductor and/or quadriceps muscle which, when attached to proximal femoral implant 80, enhance the kinematics of the hip joint. In one embodiment, at least one suture or other surgical fastener may be threaded through throughbore 101 (FIG. 17) defined by aperture 100 in lateral protrusion 94 to further facilitating holding the soft tissue structure, such as the abductor muscle, in relation to porous surfaces 106, 107, 109. Therefore, apertures 108, 98, 100 provide points from which soft tissue structures are pulled into contact with proximal femoral implant 80 and facilitate providing a sling around the hip joint to maximize hip joint function and kinematics.

Although not illustrated in FIGS. 13-17, proximal femoral implant 80 may also use at least one structure similar to washer 50, described above with respect to FIGS. 6-8, to mechanically fixate a soft tissue structure to proximal femoral implant 80 and to facilitate grasping and pulling a soft tissue structure 50 into contact with proximal femoral implant 80.

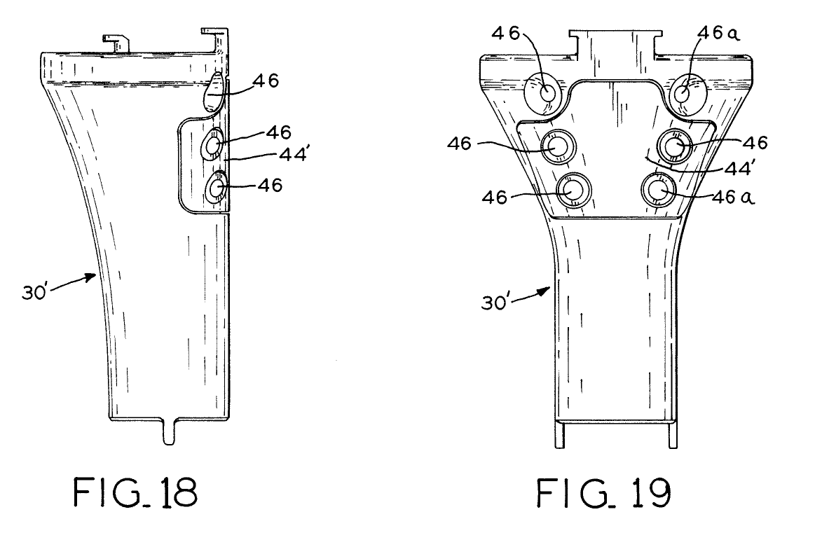

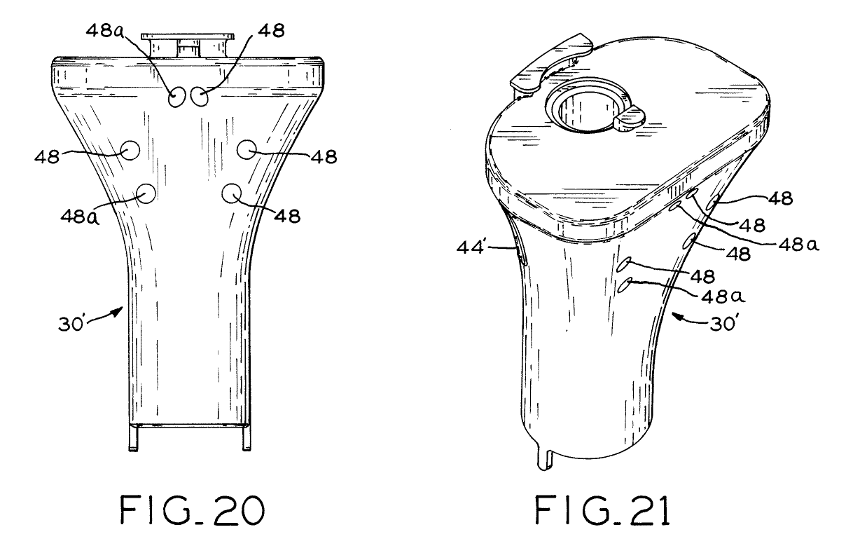

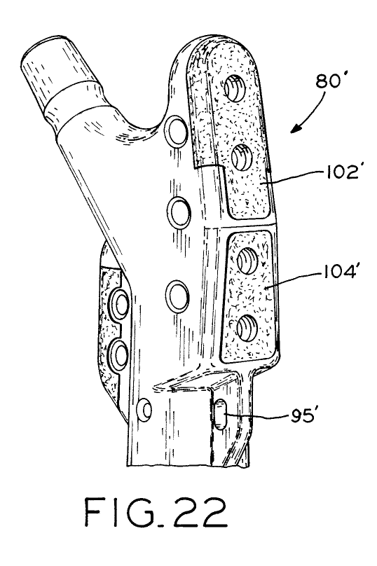

As shown in FIGS. 18-21, an alternative embodiment proximal tibial implant 30′ is shown which includes attachment plate 44′ which only surrounds some, but not all, of suture holes 46. As shown in FIG. 22, an alternative embodiment proximal femoral implant 80′ is shown which has inlaid ingrowth pads 102′, 104′, 95′.

Although the methods and apparatuses described in this disclosure describe attachment of natural soft tissue structures to the orthopaedic implants, the methods and apparatuses may also be used to secure artificial soft tissue structures to the orthopaedic implants in substantially similar manners.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims.

What is claimed is:

1. A segmental tibial orthopaedic implant, comprising:
a body; and
a porous surface attached to at least a portion of said body to facilitate attachment of soft tissue structures and/or bone to said body.

2. The segmental tibial orthopaedic implant of claim 1, further comprising at least one washer connected to said body, said washer to facilitate moving the soft tissue structures into contact with said porous surface.

3. A segmental femoral orthopaedic implant, comprising:
a body; and
a porous surface attached to at least a portion of said body to facilitate attachment of soft tissue structures and/or bone to said body.

4. The segmental femoral orthopaedic implant of claim 3, further comprising at least one washer connected to said body, said washer to facilitate moving the soft tissue structures into contact with said porous surface.
